SEQUENCE LISTING (4 sequences)

SEQ ID NO: 1 - DNA, 356 bp, Homo sapiens
  misc_feature (188)..(188): n is a, c, g, or t
ggcggggcagggtttggtgtctgtgccctttgtcaggtggcaagagctggtgtcactcacggcctgcaagaggactgcatttgcaaaaaggtgcctttctattctattgccctagggggtctccactggctgatagagaacttactcggctcccaagttactggttgatgctgttctgatcagctggngagcacctcaaggctggaatgaatgcctgtccacctttgcatccagaaaagtttttattaagcaaatgaattttcaccctcaactgtaggctatcatttctagtccagcatagaaggcttgggctattccaaccattaaacatttagatttatagtcagaagcacttg

SEQ ID NO: 2 - DNA, 356 bp, Homo sapiens
ggcggggcagggtttggtgtctgtgccctttgtcaggtggcaagagctggtgtcactcacggcctgcaagaggactgcatttgcaaaaaggtgcctttctattctattgccctagggggtctccactggctgatagagaacttactcggctcccaagttactggttgatgctgttctgatcagctggtgagcacctcaaggctggaatgaatgcctgtccacctttgcatccagaaaagtttttattaagcaaatgaattttcaccctcaactgtaggctatcatttctagtccagcatagaaggcttgggctattccaaccattaaacatttagatttatagtcagaagcacttg

SEQ ID NO: 3 - DNA, 218 bp, Homo sapiens
cccacaggagtccatttcaactctaccccatgggcaggtaaggaaactgggctctgcaagtcacaaacaagtcagcagagtcaaggagggcaggcctgacctcggccacaggtgctgcagatcagaccacattaaccaggaacttgagttgaagtccaggcggagcttgcggggagaatggctagctctctgtggagttgaaggtcgttggaggtttg

SEQ ID NO: 4 - DNA, 218 bp, Homo sapiens
cccacaggagtccatttcaactctaccccatgggcaggtaaggaaactgggctctgcaagtcacaaacaagtcagcagagtcaaggagggcaggcctgaccttggccacaggtgctgcagatcagaccacattaaccaggaacttgagttgaagtccaggcggagcttgcggggagaatggctagctctctgtggagttgaaggtggttggaggtttg

(19) United States
(12) Patent Application Publication
(10) Pub. No.: US 2023/0227854 A1
(43) Pub. Date: Jul. 20, 2023

(54) GENETIC MODIFICATION SITE

(71) Applicant: ReNeuron Limited, Pencoed, Bridgend, Wales (GB)

(72) Inventors: Randolph CORTELING, Pencoed (GB); Ruben DEOGRACIAS, Pencoed (GB)

(21) Appl. No.: 17/633,570

(22) PCT Filed: Aug. 10, 2020
(86) PCT No.: PCT/GB2020/051906

§ 371 Date: Feb. 7, 2022

(30) Foreign Application Priority Data
Aug. 9, 2019 (GB) 1911464.4

(51) Int. Cl. C12N 15/90; C12N 5/079; C12N 5/0783; C12N 5/0797; A61K 35/12; A61K 48/00

(52) U.S. Cl. CPC C12N 15/907; C12N 5/0622; C12N 5/0636; C12N 5/0623; A61K 35/12; A61K 48/005; C12N 2510/00

(57) ABSTRACT

This invention relates to genetic engineering, in particular to an insertion site for a transgene, cells comprising a transgene or other modification at that insertion site, vectors for targeting that insertion site, and methods for creating transgenic cells by insertion or other modification at that site. The insertion site, or “safe harbour locus”, is identified within the SPATA13 gene on human chromosome 13q12.12. Mammalian cells comprising a genetic modification within the SPATA13 gene on chromosome 13q12.12 are described, wherein the modification may be an insertion such as an integrated transgene. Nucleic acid molecules able and adapted to guide the insertion of a transgene to that insertion site are also described. These cells or nucleic acids may be useful in therapy.

### FIELD OF THE INVENTION

This invention relates to genetic engineering, in particular to an insertion site for a transgene, cells comprising a transgene or other modification at that insertion site, vectors for targeting that insertion site, and methods for creating transgenic cells by insertion or other modification at that site.

### BACKGROUND OF THE INVENTION

The creation of transgenic cells and animals is well-established. However, a drawback of traditional transgenic techniques is that integration of the transgene is a random event, so the transgene cannot be directed to a specific chromosomal location. Random insertion into a genome can cause problems including insertional mutagenesis and gene silencing.

Newer techniques using site-specific recombinases such as the bacteriophage P1 recombinase Cre, and gene-editing and chromosome engineering techniques using engineered endonucleases such as CRISPR, Zinc Finger Nucleases and TALENs, allow for site-specific transgene integration into the genome. However, while site specific insertion is now possible, many transgenic lines are still being created using the conventional random insertion method. One of the emerging challenges of these relatively new site-specific insertion technologies is the question of where specifically to insert the transgene. In addition to the need for stable integration, a major challenge is that the genomic environment at the integration site has a substantial influence on the expression of the transgene, and may also cause downstream changes in the expression of neighbouring genes that can also cause unwanted effects. The effect of the chromosomal insertion position upon transgene expression level is still relatively unknown. Furthermore, cells that have a therapeutic purpose must also be safe and act predictably.

A small number of sites in the human genome have been proposed to be genomic safe harbors, where transgenes can be inserted and expressed without causing significant alternations in the expression of other genetic elements (Lombardo et al., 2011). However, there are not enough known and validated ‘safe harbour’ genome loci into which an introduced recombinant gene or genes can be targeted. As noted by Papapetrou and Schambach (Mol Ther. 2016 April; 24(4): 678-684), no fully validated GSHs exist in the human genome. Accordingly, there remains a need to provide transgenic cells and animals that are stable and that provide acceptable expression of the transgene. For transgenic cells with therapeutic utility, the transgenic cell must also be safe, consistent and repeatable, for example across batches.

### SUMMARY OF THE INVENTION

The invention is based on the surprising identification of a genomic locus that is particularly favourable for genetic modification such as the insertion of a transgene. This is based on the realisation that the insertion site for the c-MycER^{TAM} transgene in the CTX0E03 cell line is safe, stable and expressed at an effective level. The integration of the transgene in the neural stem cell to create the CTX0E03 cell line was a random event, yet it has now been realised by the inventors that the specific site of integration is advantageous and allows for safe and stable insertion of any transgene into a cell. This has particular utility in the creation of cells for use in therapy.

The identified insertion site is beneficial because random insertion often doesn't work effectively and/or requires many repeated attempts to arrive at a stable insertion. The invention thus provides for a more predictable technique for genetic modification, in particular insertion of a coding sequence.

The transgenic cells of the invention find utility in a number of applications, including as research tools, in screening, as cell therapies, to produce biologics as part of a biotechnological process, and to harvest microparticles such as exosomes from the cells.

The insert can also be used to provide a means for the engineered cell to produce an exogenously introduced protein or nucleic acid drug or to enhance the features of the cell to make it more suitable for use in any of the above applications, for example in a biotechnological process. For example, the engineering can provide the ability to scale up cell culture expansion and increase the number of passages that a cell can stably be expanded in culturing ex vivo, or to contain characteristics to facilitate cell tracking/labelling or purification.

The genetic locus of the invention provides a so-called “safe-harbour” for engineering, that is safe and predictable. It was identified by the inventors in the CTX0E03 neural stem cell line. New applications of this technology are therefore provided by providing a different or a further insertion into that locus in the CTX0E03 cells, or by targeting the same locus in other (non-CTX0E03) cell types.

A first aspect of the invention provides a cell comprising a genetic modification within the SPATA13 gene, wherein:
(i) the cell is not a CTX0E03 cell; and/or
(ii) the genetic modification is not insertion of a cMYC-ER transgene.

In certain embodiments, the genetic modification is an insertion. In some embodiments the genetic modification is an integrated transgene, typically a stable integrated transgene.

In various embodiments, the insertion site may be:
a. within an intron of the SPATA13 gene;
b. within the third intron of the SPATA13 gene;
c. within third intron of a cDNA clone with Genbank accession number BX648244;
d. on chromosome 13q12.12 anywhere between nucleotides 24,083,250-400 bp from the P-terminus;
e. on chromosome 13q12.12 anywhere between nucleotides 24,083,300-350 bp from the P-terminus;
f. on chromosome 13q12.12 anywhere between nucleotides 24,083,325-335 bp from the P-terminus; or
g. on chromosome 13q12.12 between nucleotides −24,083,331-24,083,332 bp from the P-terminus.

The cell is typically mammalian, more typically human. When the cell is human, the SPATA13 gene insertion site is on chromosome 13q12.12.

In certain embodiments, the cell is a stem cell or a terminally differentiated cell. The cell may or may not be a neural stem cell, a neural stem cell from foetal cortical tissue, or from a neural stem cell line. The cell may, in other embodiments, not be a neural cell and/or not be a stem cell.

In one embodiment of the first aspect, the cell is a CTX0E03 cell engineered to replace the cMycER-transgene with a different transgene. The cMycER transgene may be replaced in full or in part, or the new transgene can simply be inserted into the existing cMycER transgene (typically such that the cMycER transgene is disrupted and the new transgene functionally replaces it). In some embodiments, the transgene is inserted before or after, for example immediately before or after, the cMyc-ER transgene or within about 100 bp of the start or end of that transgene.

According to a second aspect, cells of the first aspect are provided for use in therapy.

According to a third aspect, a cell according to the first aspect is provided for use in a biotechnological process, optionally wherein the process is the production of a stem cell, protein or microparticle such as an exosome.

According to a fourth aspect is provided a pharmaceutical composition comprising a cell according to the first aspect.

A fifth aspect of the invention provides a method of producing a transgenic cell comprising a stable integrated transgene, comprising the step of integrating the transgene at a site within the SPATA13 gene. Typically the SPATA13 gene is on chromosome 13q12.12. Typically, the transgenic cell is human. The transgene may be or comprise human or non-human sequence. This method can be applied to cells in vitro or in vivo. This aspect has particular utility in gene therapy, in particular when used in vivo.

The method of the fifth aspect can optionally comprise one or more elements of CRISPR, TALENS or other site-specific genetic modification technology. In certain embodiments of the fifth aspect, the insertion site is targeted using a construct, for example a gene therapy construct, able and adapted to guide the insertion of the exogenous transgene to the insertion site following administration to a human or animal.

A sixth aspect provides a cell obtained or obtainable from the method of the fifth aspect.

In a further aspect, the invention provides a nucleic acid molecule able and adapted to guide the insertion of a transgene to the insertion site, typically within the SPATA13 gene and optionally on chromosome 13q12.12, following administration to a human or animal. This nucleic acid may be used in therapy, for example gene therapy.

### BRIEF DESCRIPTION OF THE DRAWINGS

FIG. 1: BLAST homology for the two flanking sequences (shown as Panel A and Panel B). The top line shows the supplied sequence whereas the bottom line shows the genomic position on chromosome 13. One mismatch is evident with flanking sequence A due to a missing base call in the original sequencing. Two mismatches are evident in sequence B. These are most likely due to sequencing errors. The top line (supplied sequence) in Panel A is SEQ ID No. 1. The bottom line (genomic sequence) in Panel A is SEQ ID No. 2. The top line (supplied sequence) in Panel B is SEQ ID No. 3. The bottom line (genomic sequence) in Panel B is SEQ ID No. 4.

FIG. 2: (A) Diagram showing the 1 Mb region either side of the integration site. The distance from the chromosome 13 p-telomere is shown on the top. Known genes as indicated by the UCSC genome browser are shown (in blue in the original format). Vertical lines/boxes represent exons, fine lines with arrows in between represent introns. Those exons with half-height boxes represent untranslated exons. Superfamilies, i.e. identifiable domains/motifs, are shown (in ref in the original colour format). The bottom section shows the distribution of various repeat sequences. (B) Close up diagram of the integration site which lies within intron 3 of BX648244. CpG islands have also been displayed in this diagram (in green in the original), as are exons (in red). The UCSC genome browser-identified genes AK092754 and MGC48915 as well as BX648244 are all isoforms of the SPATA13 gene. Of particular interest for the integration site is that most of the 5′ exons are untranslated, i.e. Pre-messengers (indicated on the diagram as half-height boxes).

FIG. 3: Schematic diagram showing the surrounding area of the integration site for the transgene c-myc-ERTAM in the human Chromosome13q12.12 (GRCh38:13:24083331-24083332). Below, magnification of 1M base pair indicating the nearest genes surrounding the insertion site at the locus SPATA13.

FIG. 4: Schemes showing the use of CRISPR/Cas9 to allow the integration of a DNA in the chromosome 13. (A) Representation of the elements necessary to induce Cas9-dependent homologous recombination in chromosome 13. (B) Diagram showing homologous integration of a DOI between the positions (GRCh38) chr13:24,083,331 and chr13: 24,083,332.

### DETAILED DESCRIPTION OF THE INVENTION

The present inventors have highlighted a favourable chromosomal insertion site. This site is within the SPATA13 gene on human chromosome 13q12.12. Equivalent insertion sites in the same gene locus exist in the genomes of cells from other animal species.

In certain embodiments the insertion site provides a target locus, such as for gene therapies, to enable the insertion of genes, gene portions or other genetic elements. The expression of the insert can in some embodiments be constitutively expressed or may be conditionally-activated through use of conditionally-activated promoters known in the art. One such conditionally-active promoter is the TAM conditionally-activated promoter used in the Examples below.

In certain embodiments, one or more therapeutic genes or gene portions can be targeted to the locus using a sequence complementary to the target locus sequence identified herein. For example, a nucleic acid that is complementary to the sense or antisense strand of the locus of the invention can be provided as a guide strand for gene editing technology such as CRISPR.

In some embodiments, the locus allows for the engineering of cell lines that can be used as a GMP manufacturing source for producing biological agents or therapeutics. The biological agents or therapeutics may comprise antibodies or fragments thereof, proteins, glycoproteins, peptides, lipoproteins or the like. In some methods, the cell of the invention is provided for the production of a stem cell or microparticle. A microparticle is typically an extracellular vesicle of 30 to 1000 nm diameter that is released from a cell. It is typically limited by a lipid bilayer that encloses biological molecules. The term “microparticle” is known in the art and encompasses a number of different species of microparticle, including a membrane particle, membrane vesicle, microvesicle, exosome-like vesicle, exosome, ectosome-like vesicle, ectosome or exovesicle. Typically, the microparticle produced by the cell of the invention is an exosome.

New sources of cells can provide advantages over existing cell lines such as CHO and PerC6. Cell lines engineered to include an exogenous coding sequence at the insertion locus having a stable genome and a low risk of adversely affecting cell growth, health or toxicity are thus provided.

In one embodiment, Schwann cells are immortalised by inserting a conditional oncogene at the insertion site. These cells may then have utility in nerve repair and/or regeneration typically by supporting both axonal growth and myelination. A conditional oncogene may, for example, be C-mycER or L-mycER.

Modified cells of the invention can be used in cell therapy in humans or animals. In one embodiment, the cells of the first aspect are provided for use in treatment of a disease or inherited condition in a human or animal in need of such treatment. A method of treating a patient in need thereof is also provided, comprising administering to the patient a therapeutically effective amount of (i) transgenic cells of the invention or (ii) a nucleic acid vector able to guide the insertion of a transgene to the insertion site. The patient may also be treated by administering guide RNA (e.g. sgRNA) targeted to the insertion site, optionally together with a Cas endonuclease such as Cas9. The patient is typically human.

Processes for site-specific integration may typically involve the steps of 1) introducing a targeting vector containing a gene of interest into mammalian cells and 2) screening and selecting transfected cells with integration of the gene of interest at specific genomic locus. Site specific gene insertion is known in the art, for example as described in Nature Methods volume 10, page 13 (2013).

The invention also provides nucleic acids, vectors and gene therapy constructs, for targeting the insertion site. These typically provide improved targeting for safer and more efficacious gene therapy in humans or animals.

Modification Site

The invention identifies a particularly useful site in the human genome that can be used for genetic modification. This site will typically be used for the insertion of a nucleic acid coding for a protein of interest, as was the case in the CTX0E03 cells in which the site was identified, but other genetic modifications at this site are possible. This locus is generally referred to herein as the insertion site.

The insertion site was identified within the SPATA13 gene on human chromosome 13q12.12. In chimpanzees (Pan troglodytes) the SPATA13 gene is also located on chromosome 13, while its chromosomal location varies in other animals: chromosome 14 in the mouse (Mus musculus), chromosome 15 in the rat (Rattus Norvegicus), chromosome 17 in the Rhesus monkey (Macaca mulatta), chromosome 24 in the zebrafish (Danio rerio) and chromosome 2 in the African clawed frog (Xenopus laevis). In some embodiments the SPATA13 gene as described herein in any animal cell is targeted for modification, for example within the third intron of the SPATA13 gene. Typically the animal cell is a mammalian cell, more typically a great ape such as a chimpanzee or gorilla. More typically, the insertion site is in a human cell.

The exact location of the insertion in the CTX0E03 cells is on (GRCh38) chromosome 13q12.12 between nucleotides 24,083,331-332 bp from the P-terminus. However, it is expected that equivalent results can be obtained if a site in that general area is targeted according to the invention, for example within 10 kb, or within 5 kb, within 2.5 kb, for example within 1000 bp or within 500 bp of that specific site. In certain embodiments, the locus targeted for modification can be within an intron of the SPATA13 gene. In further embodiments, the locus is within the third intron of the SPATA13 gene. Typically, the locus is within the third intron of a cDNA clone with Genbank accession number BX648244. More specifically, the locus may be on chromosome 13q12.12 anywhere between nucleotides 24,083,250-400 bp from the P-terminus, anywhere between nucleotides 24,083,300-350 bp from the P-terminus, or anywhere between nucleotides 24,083,325-335 bp from the P-terminus.

The insertion site is referred to as “GRCh38:13: 24083331-24083332”. GRCh38 refers to the version of the human genome reference currently used by UCSC browser, as will be apparent to the skilled person.

The insertion site was identified in the neural stem cell referred to as “CTX0E03” deposited by ReNeuron Limited at the European Collection of Authenticated Cell Cultures (ECACC), Porton Down, UK and having ECACC Accession No. 04091601.

The cells of the CTX0E03 cell line are multipotent cells originally derived from 12 week human fetal cortex. The isolation, manufacture and protocols for the CTX0E03 cell line is described in detail by Sinden, et al. (U.S. Pat. No. 7,416,888 and EP1645626 B1). The CTX0E03 cells are not “embryonic stem cells”, i.e. they are not pluripotent cells derived from the inner cell mass of a blastocyst; isolation of the original cells did not result in the destruction of an embryo. In growth medium CTX0E03 cells are nestin-positive with a low percentage of GFAP positive cells (i.e. the population is negative for GFAP).

CTX0E03 is a clonal cell line that contains a single copy of the c-mycER transgene that was delivered by retroviral infection and is conditionally regulated by 4-OHT (4-hydroxytamoxifen). The C-mycER transgene is incorporated at the insertion site identified herein and expresses a fusion protein that stimulates cell proliferation in the presence of 4-OHT and therefore allows controlled expansion when cultured in the presence of 4-OHT. This cell line is clonal, expands rapidly in culture (doubling time 50-60 hours) and has a normal human karyotype (46 XY). It is genetically stable and can be grown in large numbers. The cells are safe and non-tumorigenic. In the absence of growth factors and 4-OHT, the cells undergo growth arrest and differentiate into neurons and astrocytes. Once implanted into an ischemia-damaged brain, these cells migrate only to areas of tissue damage.

The cells of the CTX0E03 cell line may be cultured in the following culture conditions:
Human Serum Albumin 0.03%
Transferrin, Human 5 μg/ml
Putrescine Dihydrochloride 16.2 μg/ml
Insulin Human recombinant 5 μl/ml
Progesterone 60 ng/ml
L-Glutamine 2 mM
Sodium Selenite (selenium) 40 ng/ml

Plus basic Fibroblast Growth Factor (10 ng/ml), epidermal growth factor (20 ng/ml) and 4-hydroxytamoxifen 100 nM for cell expansion. The cells can be differentiated by removal of the 4-hydroxytamoxifen. Typically, the cells can either be cultured at 5% CO₂/37° C. or under hypoxic conditions of 5%, 4%, 3%, 2% or 1% O₂. These cell lines do not require serum to be cultured successfully. Serum is required for the successful culture of many cell lines, but contains many contaminants. An advantage of the CTX0E03 neural stem cell line, or any other cell line that does not require serum, is that the contamination by serum is avoided.

Genetic Modification

The invention provides for modification of a cell at the genetic locus (the “insertion site”) described herein.

Typically the modification will be the insertion of a nucleic acid. A nucleic acid that is inserted into the insertion site may be exogenous to the cell. Alternatively, the nucleic acid to be inserted may naturally be found at a different locus within the cell. Typically the inserted nucleic acid will be DNA, but may also be RNA, a hybrid of DNA or RNA or comprising another nucleic acid, non-standard nucleic acid or polymer thereof.

The inserted nucleic acid may be a coding or a non-coding sequence. In typical embodiments, the nucleic acid sequence is a coding sequence of DNA that leads to the expression of a protein of interest. The inserted nucleic acid may be from the same species as the cell to be modified (an allogenic insertion) or not (a xenogenic insertion). The inserted sequence may comprise one or more control sequences, such as promoters, necessary to ensure that the inserted sequence is functional. For a coding sequence, a functional sequence is one that can successfully be transcribed.

In some embodiments, the inserted nucleic acid is a transgene. In certain embodiments, for example, the inserted transgene can improve recombinant protein expression in the cell. Examples of such transgenes include, but are not limited to, EBNA-1, GS, XBP1, or ERO-La (Hunter M, et al. 2018. Current Protocols in Protein Science). In other embodiments, the inserted transgene confers immortality or conditional immortality upon the cell. Other embodiments insert a new or an enhanced function onto the cell, for example a new cell surface receptor or new enzymatic or structural function. One such engineered cell type is a chimeric antigen receptor engineered into an immune cell such as a T cell or a natural killer cell. CAR-T and CAR-NK cells can therefore be produced by inserting the chimeric antigen receptor into the insertion site of the invention, in a T cell (e.g. CD8+ cytotoxic T lymphocyte) or NK cell. CAR-T and CAR-NK cells are well-known for use in therapy, for example in methods of treating cancer such as a blood cancer.

The result of the insertion will be to create a sequence (and therefore a cell) that does not exist in nature, i.e. has a sequence inserted into the locus that is not present in the unmodified chromosome.

The inserted sequence may itself be an artificial sequence, for example created recombinantly, that does not exist in nature. An example is a fusion construct of two sequences, for example that encodes a fusion protein. An example of a fusion protein is a chimeric antibody. Other non-naturally occurring proteins include humanised antibodies comprising a mixture of non-human CDR and human constant region and variable framework sequences, and chimeric antigen receptors typically comprising an antibody-derived antigen-binding extracellular portion and a T cell receptor transmembrane and intracellular portion. CAR-T cells produced using CRISPR to target the CAR coding sequence to the insertion site of the invention is an exemplary embodiment.

In one aspect of the invention the cell that is modified is a mammalian cell. In some embodiments the mammalian cell is a either a human cell or an animal cell. The cells may be from the muscle, epithelial, connective or nervous systems. Examples of mammalian or human cells include, but are not limited to, somatic cells, neural cells, muscle cells, red blood cells, white blood cells, immune cells, T cells, CD4+ T cells, CD8+ T cells, B cells, bone cells, fat cells, skin cells, cardiac cells, pancreatic cells and liver cells. In some embodiments, the cell is a stem cell, a progenitor cell, a multipotent cell, a pluripotent cell, an induced pluripotent stem (iPS) cell, or a non-stem (terminally differentiated) cell.

When used in therapy, the cells may be allogeneic or autologous to the patient. Autologous cell therapies are becoming well established, such as autologous CAR-T cell therapies for example.

In some embodiments, the locus allows for the engineering of cell lines that can be used as a GMP manufacturing source for producing biological agents or therapeutics. Examples of human cell lines include, but are not limited to, HeLa, HEK293 cells, U2OS, HCT 116, MDA-MB-231, MDA-MB-435, U87, U251, Raji cell, Jurkat cells, PC3, MCF-7, Saos-2, HL-60, and LNCAP cells. Examples of animal (mammalian) cell lines include, but are not limited to, CHO, BHK, NS0, SP2/0, YB2/0, COS, MDCK, MSC-1, CAD, P19, NIH 3T3, L929, N2a, and J558L cells. CHO cells are commonly used for the production of biologic drugs and are typical cells for those embodiments.

Immortalisation and Conditional Immortalisation

In certain embodiments, the nucleic acid to be inserted at the insertion site is a sequence that confers immortality upon the cell. For example, the nucleic acid to be inserted may encode an immortalisation factor. Immortalisation factors are well known in the art and include one or more viral oncogenes and hTERT (Human telomerase reverse transcriptase), or a combination thereof. Viral oncogenes include the adenoviral E1A/E1B genes, the simian virus 40 large T antigen (SV40 Tag) and the human papillomavirus 16 (HPV16) E6/E7 genes.

In other embodiments, the inserted sequence confers conditional immortality upon the cell, wherein the expression of an immortalisation factor can be regulated. Conditional-immortalisation factors are known in the art and include the temperature-sensitive SV40 Tag, C-MycER or L-mycER.

In a conditionally immortalised cell, the expression of an immortalisation factor can be regulated without adversely affecting the production of therapeutically effective stem cells. This may be achieved by introducing an immortalisation factor which is inactive unless the cell is supplied with an activating agent. Such an immortalisation factor may be a gene such as c-mycER. The c-MycER gene product is a fusion protein comprising a c-Myc variant fused to the ligand-binding domain of a mutant estrogen receptor. C-MycER only drives cell proliferation in the presence of the synthetic steroid 4-hydroxytamoxifen (4-OHT) (Littlewood et al. 1995). This approach allows for controlled expansion of neural stem cells in vitro, while avoiding undesired in vivo effects on host cell proliferation (e.g. tumour formation) due to the presence of c-Myc or the gene encoding it in the neural stem cell line.

CTX0E03 is an example of a conditionally immortalised stem cell line. Exemplary conditionally-immortalised cell lines include the CTX0E03, STR0C05 and HPC0A07 neural stem cell lines, which have been deposited by the applicant of this patent application, ReNeuron Limited, at the European Collection of Animal Cultures (ECACC), Vaccine Research and Production laboratories, Public Health Laboratory Services, Porton Down, Salisbury, Wiltshire, SP4 0JG, with Accession No. 04091601 (CTX0E03); Accession No. 04110301 (STR0C05); and Accession No. 04092302 (HPC0A07). The derivation and provenance of these cells is described in EP1645626 B1 and U.S. Pat. No. 7,416,888.

In one embodiment, the invention provides a cell that is not CTX0E03 but that comprises the same conditionally-immortalising transgene inserted at the same site (i.e. the insertion locus described herein). This may be a stem cell such as a neural stem cell, a mesenchymal stem cell or a haematopoietic stem cell, or a non-stem cell such as a differentiated cell. Other cells are described elsewhere herein.

An example of a differentiated cell that can be modified to contain the c-mycER transgene at the insertion locus is a Schwann cell. For the avoidance of doubt, one aspect of the invention therefore provides a Schwann cell having c-MycER (or alternatively L-Myc-ER) inserted at the insertion site to make conditionally-immortalised Schwann cells.

Targeting of Integration Site

Site-specific insertion of nucleic acid into the genome can be achieved by genome editing tools such as meganucleases, zinc finger nucleases (ZFN), transcription activator-like effector nucleases (TALENs) and clustered regulatory interspaced short palindromic repeats (CRISPR)-associated (Cas) RNA guided nucleases (Gaj T, et al. 2013. Trends in Biotechnology). These are all well-known methods in the field of genome editing. Upon site-specific DNA double-strand breaks (DSBs) or single-strand breaks (SSBs) induced by engineered nucleases, the target locus typically will be repaired by one of two major DNA damage repair pathways: nonhomologous end-joining (NHEJ) or homology-directed repair (HDR). The skilled reader will appreciate how to target an identified genomic locus using these techniques.

Meganucleases, ZFNs and TALENs have been used extensively for genome editing. Meganucleases are engineered versions of naturally occurring restriction enzymes. These enzymes typically have extended DNA recognition sequences (e.g. 14-40 bp). ZFNs and TALENs are artificial fusion proteins composed of an engineered DNA binding domain fused to a nonspecific nuclease domain. Zinc finger and TALE repeat domains with customised specificities can be joined together into arrays that bind to extended DNA sequences (Sander J D, and Joung K. 2014. Nature Biotechnology).

The CRISPR-Cas9 system also allows for targeted editing of DNA. The system is targeted to the DNA via association with a guide RNA (gRNA) molecule, which binds to the targeted DNA through base complementarity and enables precise DNA cleavage. gRNAs are around 100 nt in length. The targeting specificity of the CRISPR-Cas9 system is determined by a 17-21 nucleotide sequence at the 5′ end of the gRNA, which is complementary to the target site. In a S. pyogenes CRISPR-Cas9 system, the desired target sequence must immediately precede a 5′ PAM motif (NGG).

HDR is desired repair pathway for insertion of nucleic acid into the genome of cell. Wild-type Cas9 enzymes can be used to introduce double-strand breaks in the DNA and mutant Cas9 enzymes have been generated that introduce single-stranded breaks. In some methods, to encourage HDR (which occurs at a lower frequency than NHEJ) mutant Cas9 enzymes can be used, which only make single-stranded breaks. These mutant Cas9 enzymes can be paired with gRNAs that target the sense strand and antisense strand of DNA of the insertion site to introduce targeted double-strand breaks, which are repaired by HDR in the presence of the exogenous nucleic acid to be inserted (Ran F A, et al. 2013. Cell; Koch B, et al. 2018. Nature Protocols).

Insertion of nucleic acid into the target integration site can be achieved by designing a donor plasmid harbouring short homology arms, which the insertion site of the invention. The homology arms are typically between 100 and 2000 bp in length (Koch B, et al. 2018. Nature Protocols).

In some embodiments, the invention provides a gRNA, typically an isolated gRNA, designed to hybridise to the insertion site identified herein. Design of CRISPR guide RNAs is well-known in the art. The 17-21 nt sequence at the 5′ end of the gRNA that is complementary to the target site is typically at least 70% identical, at least 80% identical, at least 90% identical, at least 95% identical, at least 99% identical or is perfectly complementary to a 17-21 nt portion of the insertion site identified herein. In some embodiments, the ctarget-complementary region of the gRNA is complementary to a region in an intron of SPATA13, typically the third intron of SPATA13.

In some embodiments, the key gRNA region is complementary to a sequence on chromosome 13q12.12.

In certain embodiments, the 17-21 nt sequence at the 5′ end of the gRNA is at least 70% identical, at least 80% identical, at least 90% identical, at least 95% identical, at least 99% identical or is perfectly complementary to a 17, 18, 19, 20 or 21 nt sequence spanning nucleotides 24,083,331-24,083,332 bp from the P-terminus on human chromosome 13q12.12. In certain embodiments the complementary region is 20 nt in length.

In some embodiments, the invention provides a composition or kit comprising a DNA vector encoding Streptococcus pyogenes Cas9 endonuclease and a single guide RNA as discussed above.

The insertion site was identified in human cells. In some embodiments, the insertion locus in non-human cells can be identified by hybridisation of a gRNA used by the CRISPR-Cas9 system of genome editing, that is designed to target the insertion site in the CTX cells as discussed above. Without being bound by theory, it is anticipated that a gRNA designed to hybridise with the insertion site in CTX0E03 cells will hybridise with similar insertion sites in different mammalian cells. Potential insertion sites in mammalian cells can therefore be identified by the gRNA hybridising with the DNA insertion site identified herein.

Hybridisation will usually be carried out under stringent conditions, known to those in the art, chosen to reduce the possibility of non-complementary hybridisation. Examples of suitable hybridising conditions are disclosed in Nucleic Acid Hybridisation: A Practical Approach (B. D. Hames and S. J. Higgins, editors IRL Press, 1985). An example of stringent hybridisation conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulphate, and 20 μg/ml denatured, sheared salmon sperm DNA, followed by washing in 0.1×SSC at about 65° C.

Pharmaceutical Compositions

The engineered cells and the vectors and nucleic acids of the invention are useful in therapy and can therefore be formulated as a pharmaceutical composition. A pharmaceutically acceptable composition typically includes at least one pharmaceutically acceptable carrier, diluent, vehicle and/or excipient in addition to the product of the invention. An example of a suitable carrier is Ringer's Lactate solution. A thorough discussion of such components is provided in Gennaro (2000) Remington: The Science and Practice of Pharmacy, 20th edition, ISBN: 0683306472.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain minor amounts of pH buffering agents. The composition may comprise storage media such as Hypothermosol®, commercially available from BioLife Solutions Inc., USA. Examples of suitable pharmaceutical carriers are described in “Remington's Pharmaceutical Sciences” by E W Martin. Such compositions will contain a prophylactically or therapeutically effective amount of a prophylactic or therapeutic stem cell preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, frozen preparations, liquid solutions or suspensions, injectable and infusible solutions. The pharmaceutical composition is preferably injectable.

Pharmaceutical compositions will generally be in aqueous form. Compositions may include a preservative and/or an antioxidant.

To control tonicity, the pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition is preferably sterile. The composition is preferably non pyrogenic.

In a typical embodiment, cells are suspended in a composition comprising 1, 2, 3, 4, 5, 6, 7, 8, 9, 10, or more excipients selected from 6 hydroxy-2,5,7,8-tetramethylchroman-2-carboxylic acid (Trolox®), Na+, K+, Ca2+, Mg2+, Cl−, H2P04−, HEPES, lactobionate, sucrose, mannitol, glucose, dextron-40, adenosine and glutathione. In one embodiment the composition comprises all of these excipients. Typically, the composition will not include a dipolar aprotic solvent, e.g. DMSO. Suitable compositions are available commercially, e.g. HypoThermasol®-FRS. Such compositions are advantageous as they allow the cells to be stored at 4° C. to 25° C. for extended periods (hours to days) or preserved at cryothermic temperatures, i.e. temperatures below −20° C. The stem cells may then be administered in this composition after thawing.

The invention is further described with reference to the following non-limiting examples.

### Examples

A) Safe Harbour Locus for Gene Expression

Introduction

The insertion of transgenes for gene over-expression in human cells requires the use of safe harbour loci (SHL) that do not affect the gene expression pattern of the target cell.

So far, only 3 SHL have been identify in human cells, AAVS1 in chromosome 19, CCR5 and ROSA26 both in chromosome 3. Both CCR5 and AAVS1 are located in areas containing several genes, including cancer-related genes that can be dysregulated by the integrated transgenes. ROSA26 is also located near genes and there have been no additional studies confirming the utility or safety of this locus in human cells.

Therefore, the discovery of new SHL would be highly beneficial for the safe over-express of transgenes for therapeutic purposes.

Identification of a SHL at 1312.12 (GRCh38:13:24,083,331-24,083,332)

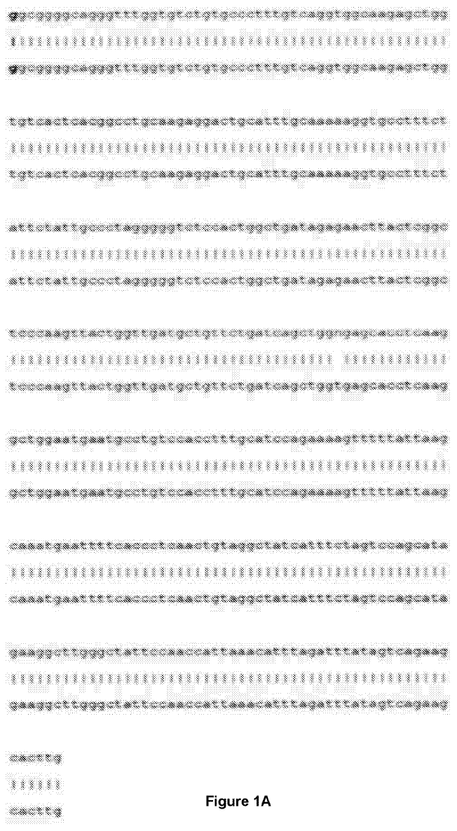

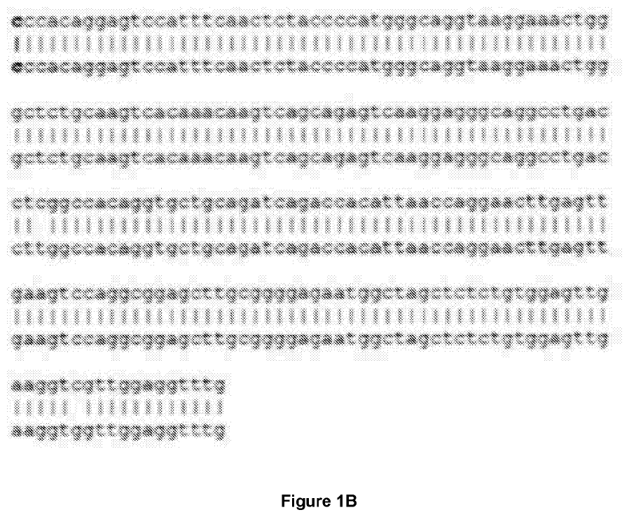

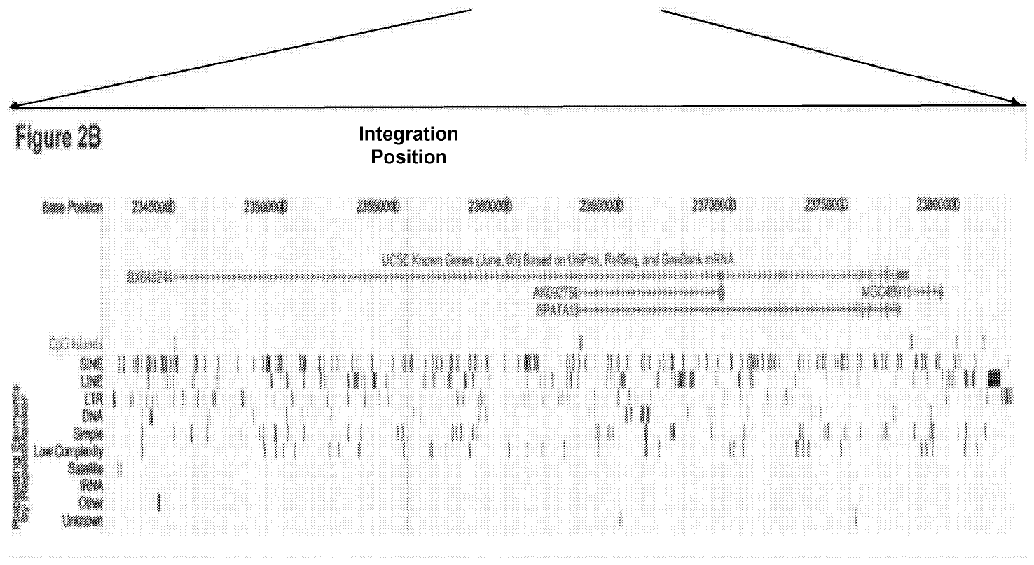

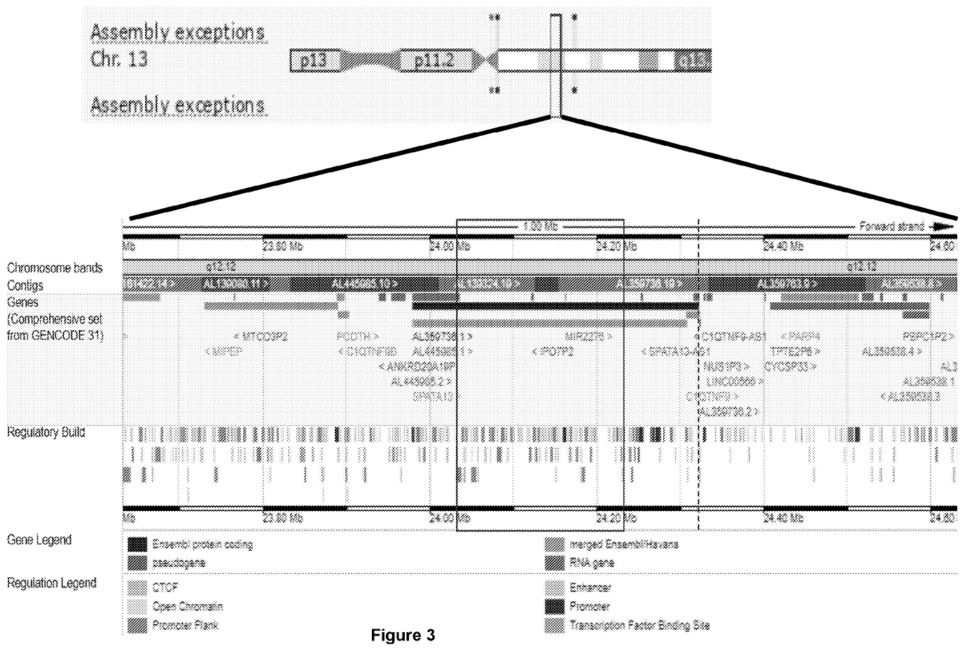

The generation of the therapeutic human cell line CTX0E03 included its immortalization by over-expression of the recombinant gene c-myc-ER^{TAM}. This was achieved by the use of a retrovirus that randomly inserted one single copy of the gene in a specific position of chromosome 13. We have mapped this region (GRCh38:13:24,083,331-24,083,332) and we have located it in an intron of the gene SPATA13. We have not observed any gene in its proximity (<180 kb) (FIGS. 1 and 3 and Table 2) and therefore it is very unlikely that any gene could be altered by this insertion or any event of chromatin remodelling due to this insertion. As the c-myc-ER is still active in our CTX0E03 cell line, this is a clear indication that the conformation of the chromatin is in an open stage that allows the continuous expression of the transgene. None of the genes identified in a region of 1 M base pair surrounding the area of insertion have been identified as cancer-related genes.

We therefore believe that this specific site on chromosome 13 has the fundamental characteristics of a SHL and can be targeted in any human cell to insert any gene for gene therapy using recombination techniques such as non-protein dependent homologous recombination, TALEN nucleases or CRISPR/Cas9 nucleases.

Cas-Targeting of SHL

For example, the use of Cas9 together a small piece of complementary RNA known as guide RNA (sgRNA) can be used to cleave any specific location in the DNA and insert any desired DNA sequence.

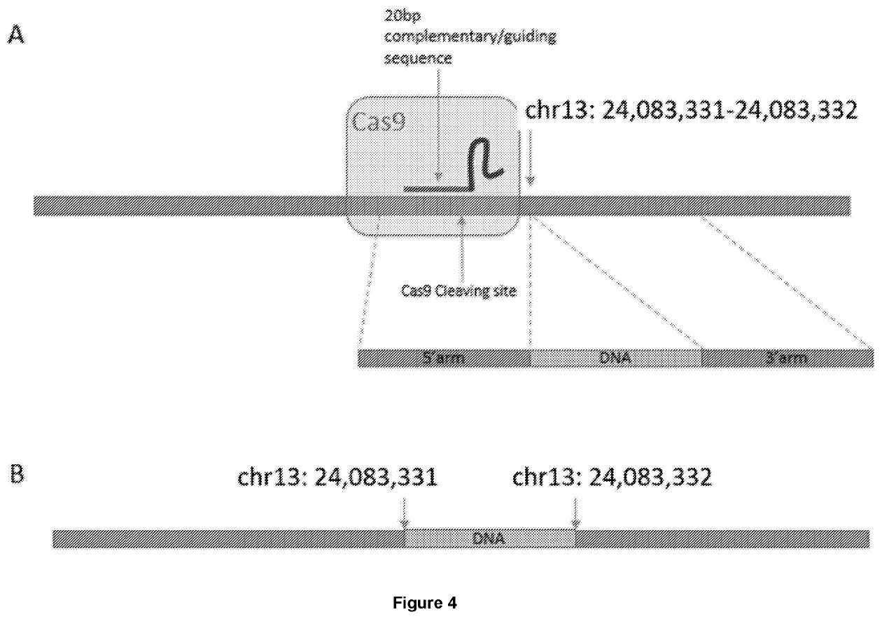

In order to trigger this specific recombination we will use a sgRNA/sgRNAs complementary to a region close to the position on GRCh38:13:24,083,331-24,083,332. That sgRNA will be delivered to the cell together with Cas9 as an expression vector or as a Ribonucleoprotein complex (RNP) by transfection, transduction, electroporation or any other way of delivering DNAs, RNAs and/or proteins. The DNA of interest (DOI), that will be also delivered together Cas9 and the sgRNA/sgRNAs, will be flanked by a 5′arm complementary to the position chr13: 24,083,331 and a 3′ arm complementary to the position chr13: 24,083,332 (FIG. 4A).

Once in the nucleus, the RNP complex will excise the chromosomal DNA in the position targeted by the sgRNA, what will trigger the DNA repairing machinery. The presence of the DOI with the complementary arms to the chromosome 13 will be recognised by the recombination machinery of the cell and inserted in the complementary region (FIG. 4B).

This will allow the insertion of the DOI between the positions chr13: 24,083,331 and chr13: 24,083,332 in any human cell line.

B) Analysis of the Transgene Insertion Site in CTX0E03

1 Introduction

The CTX0E03 is a neural stem cell line derived from primary fetal neural stem cells following transduction with a conditional immortalizing gene, cmycER^{TAM}. The transgene was engineered into a retroviral vector, pLNC-X (Clontech) and cells transduced using a retrovirus generated using the packaging cell line TEFLY-A. Insertion of the transgene into the target cells genome is by random integration via the viral LTR. For the CTX0E03 cell line we have identified the site of genomic integration for the mycER^{TAM} to be within chromosome 13. The aim of this report is to assess the possibility of insertional mutagenesis as a result of transgene integration.

Summary

A bio-informatic analysis of the insertion site and surrounding genome has indicated that the risk of oncogenic activation as a consequence of insertional mutagenesis in the CTX0E03 cell line is very low.

CTX0E03 Insertion Site

An investigation was undertaken into the retroviral insertion site for the CTX0E03 neural stem cell line. The study involved a bioinformatics analysis in order to assess the possibility that the retroviral insertion might cause the cell line to be tumorogenic as a consequence of insertional mutagenesis.

Insertional mutagenesis is a well-documented phenomenon. Retroviruses insert apparently at random into chromosomal DNA sequences. As a consequence they disrupt the existing DNA sequence. Thus they have the potential to mutate any genes into which they might insert. An example of this phenomenon causing concern was the incidence of leukaemia in three children on an X-SCID gene therapy trial utilising retroviral vectors in France (see: Science. 2005 Mar. 11; 307: 1544-5). Since the CTX0E03 cell line was generated using a retroviral vector, it is important to consider the possibility that insertional mutagenesis and oncogenic activation might occur.

Three hypotheses were investigated in this study:
1. That the provirus had inserted within a gene with oncogenic potential, in a manner likely to activate this potential;
2. That the provirus had inserted into or near an endogenous retrovirus, thereby causing activation or recombination; and
3. That the virus had inserted sufficiently close to a known oncogene that activation of the oncogene might result.

2. Location of Insertion Site—SPATA13

The flanking sequences supplied for the c-Myc-ER^{TAM} allowed the positioning of the insert to chromosome 13q12.12 between nucleotides −24,083,331-24,083,332 (GRCh38) bp (from the P-terminus)—see FIG. 1.

Retroviruses are thought to insert preferentially into intronic sequences, and that is the case here. The integration site is located within the third intron of a cDNA clone with accession number BX648244. This clone represents a splice variant of the gene SPATA13 or Spermatogenesis associated 13. The first 16 exons, which includes the two exons which flank the integration site, are ‘premessenger’ sequences, i.e. they are non-coding.

SPATA13 structure and function was investigated through bioinformatics analysis.

2.1 Structure

SPATA13 is expressed in a large number of tissues. Alternative splicing produces 18 different transcripts. The gene contains 37 introns together with 8 probable alternative promoters. It has a very long 3′ UTR. The premessenger has up to 16 exons and covers 342 kb.

2.2 Homology

Amino acid homology searches via BLAST (www.ncbi.nlm.nih.gov/BLAST) showed a 62% homology to ARHG4 (Rho guanine nucleotide exchange factor 4 [APC-stimulated guanine nucleotide exchange factor]) which acts as guanine nucleotide exchange factor (GEF) for RhoA and RAC1 GTPases. Binding of APC may activate RAC1 GEF activity. The APC-ARHG4 complex seems to be involved in cell migration as well as in E-cadherin-mediated cell-cell adhesion.

2.3 Motifs (Descriptions Edited from Interpro)

The Complement C1q protein motif is found in 3 isoforms from this gene. C1q is a subunit of the C1 enzyme complex that activates the serum complement system.

The Collagen triple helix repeat motif is found in 6 isoforms from this gene. The sequence is predominantly repeats of the G-X-Y and the polypeptide chains form a triple helix. The first position of the repeat is glycine, the second and third positions can be any residue but are frequently proline and hydroxyproline.

The Pleckstrin-like motif is found in 4 isoforms from this gene. The ‘pleckstrin homology’ (PH) domain is a domain of about 100 residues that occurs in a wide range of proteins involved in intracellular signaling or as constituents of the cytoskeleton. The function of this domain is not clear, several putative functions have been suggested: —binding to the beta/gamma subunit of heterotrimeric G proteins, —binding to lipids, e.g. phosphatidylinositol-4,5-bisphosphate, —binding to phosphorylated Ser/Thr residues, —attachment to membranes by an unknown mechanism. It is possible that different PH domains have totally different ligand requirements.

The OH motif is found in 4 isoforms from this gene. The Rho family GTPases Rho, Rae and CDC42 regulate a diverse array of cellular processes. Like all members of the Ras superfamily, the Rho proteins cycle between active GTP-bound and inactive GDP-bound conformational states. Activation of Rho proteins through release of bound GDP and subsequent binding of GTP, is catalyzed by guanine nucleotide exchange factors (GEFs) in the Dbl family. The proteins encoded by members of the Dbl family share a common domain of about 200 residues (designated the Dbl homology or DH domain) that has been shown to encode a GEF activity specific for a number of Rho family members. In addition, all family members possess a second, shared domain designated the pleckstrin homology (PH) domain. The PH domain is invariably located immediately C-terminal to the DH domain and this invariant topography suggests a functional interdependence between these two structural modules. Biochemical data have established the role of the conserved DH domain in Rho GTPase interaction and activation, and the role of the tandem PH domain in intracellular targeting and/or regulation of DH domain function. The DH domain of Dbl has been shown to mediate oligomerization that is mostly homophilic in nature. In addition to the tandem DH/PH domains Dbl family GEFs contain diverse structural motifs like serine/threonine kinase, RBD, PDZ, RGS, IQ, REM, Cdc25 RasGEF, CH, SH2, SH3, EF, spectrin or Ig. The DH domain is composed of three structurally conserved regions separated by more variable regions. It does not share significant sequence homology with other subtypes of small G-protein GEF motifs such as the Cdc25 domain and the Sec7 domain, which specifically interact with Ras and ARF family small GTPases, respectively, nor with other Rho protein interactive motifs, indicating that the Dbl family proteins are evolutionarily unique.

The SH3 motif (src Homology-3) is found in 6 isoforms from this gene. SH3 domains are small protein modules containing approximately 50 amino acid residues. They are found in a great variety of intracellular or membrane-associated proteins for example, in a variety of proteins with enzymatic activity, in adaptor proteins that lack catalytic sequences and in cytoskeletal proteins. The function of the SH3 domain is not well understood but they may mediate many diverse processes such as increasing local concentration of proteins, altering their subcellular location and mediating the assembly of large multiprotein complexes.

The Variant SH3 motif is found in 5 isoforms from this gene. SH3 (Src homology 3) domains are often indicative of a protein involved in signal transduction related to cytoskeletal organisation.

2.4 Cellular Location

NCBI Locuslink suggests functions in phosphate transport, cell adhesion and protein binding. Locuslink predicts a cytoplasmic location whereas Psort predicts a nuclear localisation. Different localisations may apply to different isoforms.

2.5 Conclusion

SPATA13 is a gene of no known function. Its structure suggests tentatively that it might be a guanine exchange factor, but based on bioinformatics alone this is a very preliminary conclusion. This would not a priori suggest SPATA13 as a likely oncogene, or a gene with any tumorogenic function.

We can conclude that the retroviral insertion does not disrupt any protein coding sequence, or any non-coding untranslated sequences. Neither are any splice-donor or -acceptor sites disrupted. Thus, we conclude that it is highly unlikely that any mutated protein is produced from this locus, or that any disrupted or truncated transcripts might emerge. There is a possibility that RNA processing might be affected. It is conceivable that enhancer sequences are affected by the insertion since they are known on occasions to be located in intronic sequences. Nonetheless, since the part of the gene in which the insertion takes place is a dispersed 5′ region between non-coding exons, such an effect appears unlikely.

We conclude that oncogenic activation of SPATA13 as a consequence of this insertion event is very unlikely.

3 Repeat Sequences

TABLE 1
Repeatmaster results for the +/−2.5 kb sequence flanking the integration
site. Position on chromosome 13 together with distance from integration site
are shown. The size of the element and percentage sequence divergence together
with percentage of nucleotide deletions and insertions are also shown.

| | | Distance | | | | | | |
|---|---|---|---|---|---|---|---|---|
| Chr13 | Chr13 | from | | | | | | |
| position | position | Integration | | | | | | |
| Begin | end | Site | Size | % | % | % | | class/ |
| (bp) | (bp) | (bp) | (bp) | div | del | ins | repeat | family |
| 23,553,456 | 23,553,475 | 1,997 | 19 | 0 | 0 | 0 | (TTTTA)n | Simple_repeat |
| 23,553,653 | 23,553,836 | 1,636 | 183 | 22 | 2 | 1 | AluJo | SINE/Alu |
| 23,553,843 | 23,553,867 | 1,605 | 24 | 0 | 0 | 0 | AT_rich | Low_complexity |
| 23,554,013 | 23,554,183 | 1,289 | 170 | 28 | 8 | 3 | L1M5 | LINE/L1 |
| 23,554,239 | 23,554,425 | 1,047 | 186 | 32 | 9 | 4 | MIRb | SINE/MIR |
| 23,554,678 | 23,554,993 | 479 | 315 | 0 | 0 | 0 | AluYb8 | SINEJAlu |
| 23,556,883 | 23,557,002 | 1411 | 119 | 11 | 0 | 2 | FLAM_C | SINE/Alu |
| 23,557,037 | 23,557,122 | 1,565 | 85 | 40 | 0 | 1 | L3 | LINE/CR1 |
| 23,557,262 | 23,557,565 | 1,790 | 303 | 13 | 0 | 1 | AluSx | SINE/Alu |
| 23,557,576 | 23,557,668 | 2,104 | 92 | 25 | 16 | 2 | L3 | LINE/CR1 |

The insertion site and up- and down-stream chromosomal regions were analysed to discover the presence of repeat sequences—i.e. sequences that might harbour endogenous retroviruses or transposable elements.

Only two types of transposable elements were found within the ±2.5 kb region of the integration site: long interspersed repetitive elements (LINEs) and short interspersed repetitive elements (SINES).

LINEs are 6 kb long transposons which harbour an internal polymerase II promoter and encode two open reading frames (ORFs). Of the LINEs, only LINE1 is active in humans. Upon translation, a LINE RNA assembles with its own encoded proteins and moves to the nucleus, where an endonuclease activity makes a single-stranded nick and the reverse transcriptase uses the nicked DNA to prime reverse transcription from the 3′ end of the LINE RNA Reverse transcription often fails to proceed to the 5′ end, resulting in mostly truncated, non-functional insertions. Most LINE-derived repeats are therefore short, with an average size of 900 bp for all LINE1 copies (IHGSC, 2001). All of the LINE repeats close to the c-mycER^{TAM} integration site are truncated forms (maximum size=170 bp) and have accumulated sequence mutations through evolution. They are therefore not active.

SINEs are short (100-400 bp) elements harbouring an internal polymerase III promoter but do not encode a protein. The major SINE element in this region is Alu, followed by a MIR element (inactive) and a FLAM_C element (fossil Alu monomer—inactive). Over 10% of the human genome comprises of Alu repeats which tend to associate themselves in gene rich areas (IHGSC, 2001). The only active SINEs are Alu elements. As they do not encode any proteins, they are reliant on L 1 machinery for retrotransposition. Many SINEs are positioned to share the 3′ end of LINE elements (Okada et al., 1997), perhaps to aid or as a consequence of retrotransposition.

None of the SINE elements in the examined region are associated with functionally active 3′ ends of LINE repeats.

We conclude that there are no endogenous retroviruses in the vicinity of the integrated provirus. Activation of endogenous retroviruses as a consequence of integration therefore appears to be a highly unlikely outcome.
International Human Genome Sequencing Consortium (2001). Initial sequencing and analysis of the human genome. Nature 409: 860-921.
Okada, N., Hamada, M., Ogiwara, I. & Ohshima, K. SINEs and LINEs share common 39 sequences: a review. Gene 205, 229±243 (1997).

4 Neighbouring Genes

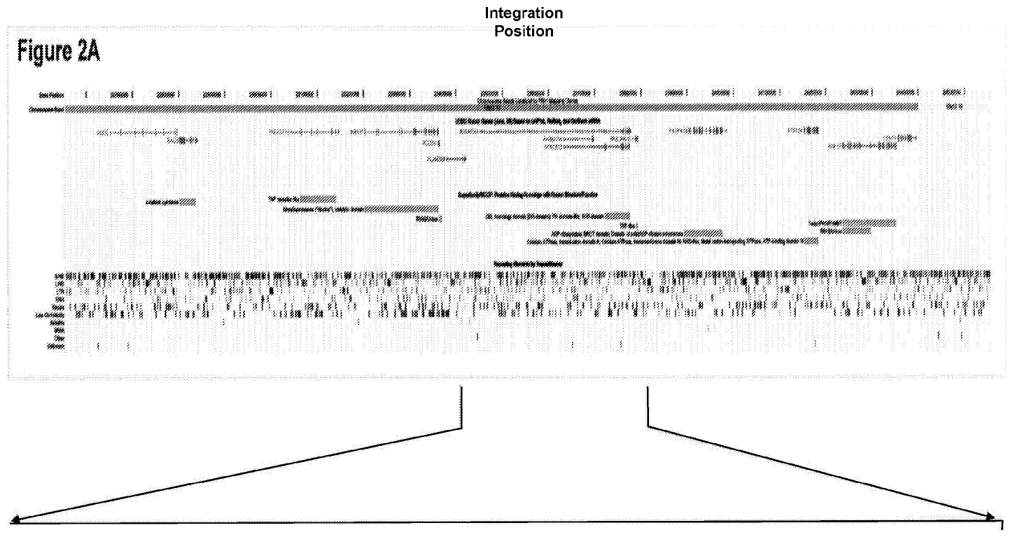

The locus of integration was analysed to discover what other genes lay in the vicinity of the integrated provirus. There are 10 known genes within a ±1 Mb region of the integration site in addition to SPAT A 13 (see table 2 and FIG. 2). An analysis was performed for each of these.

4.1 SGCG (Distance=758,167 bp)

SGCG or Gamma-sarcoglycan encodes only one transcript which is expressed in skeletal and heart muscle tissue. It is a constitutive protein of the dystrophin-glycoprotein complex (DGC), a group of proteins which span the sarcolemma and bind actin to the extracellular matrix of muscle cells (Noguchi et al. 1995). Deficiencies in SGCG are responsible for a specific form of girdle muscular dystrophy or sarcoglycanopathy, primarily involving wasting of shoulder and girth muscles along with calf muscle hypertrophy (Crosbie et al. 2000). Similarly, overexpression of gamma-sarcoglycan in mice produces severe muscular dystrophy, with greatly reduced muscle mass and early lethality (Zhu et al. 2001).
Crosbie, R. H.; Lim, L. E.; Moore, S. A.; Hirano, M.; Hays, A. P.; Maybaum, S. W.; Collin, H.; Device, S. A.; Stolle, C. A.; Fardeau, M.; Tome, F. M. S.; Campbell, K. P (2000) Molecular and genetic characterization of sarcospan: insights into sarcoglycan-sarcospan interactions. Hum. Malec. Genet. 9: 2019-2027.
Noguchi, S.; McNally, E. M.; Ben Othmane, K.; Hagiwara, Y.; Mizuno, Y.; Yoshida, M.; Yamamoto, H.; Bonnemann, C. G.; Gussoni, E.; Denton, P. H.; Kyriakides, T.; Middleton, L.; Hentati, F.; Ben Hamida, M.; Nonaka, I.; Vance, J. M.; Kunkel, L. M.; Ozawa. E. (1995) Mutations in the dystrophin-associated protein gamma-sarcoglycan in chromosome 13 muscular dystrophy. Science 270: 819-821.
Zhu X, Hadhazy M, Groh M E, Wheeler M T, Wellmann R, McNally E M. (2001) Overexpression of gamma-sarcoglycan induces severe muscular dystrophy. Implications for the regulation of Sarcoglycan assembly. J Biol Chem. 2001 Jun. 15; 276(24):21785-90. Epub 2001 Apr. 3.

TABLE 2
Summary of known genes within a +/−1 Mb region of the integration site. The chromosomal
position, the strand and calculated distance from the integration site are also provided.

| | | | | Distance | |
|---|---|---|---|---|---|
| | | | | from | |
| Start | End | | Gene | integration | 4.1.1 |
| (kb) | (kb) | Strand | Name | site (bp) | 4.1.2 Description |
| 22,653 | 22,797 | + | SGCG | 758,167 | Gamma-sarcoglycan (Gamma-SG) (35 kDa |
| | | | | | dystrophin- associated glycoprotein) |
| | | | | | (35DAG) |
| 22,906 | 22,801 | − | SACS | 649,642 | Spastic ataxia of Charlevoix-Saguenay |
| | | | | | (SACSIN) |
| 23,043 | 23,148 | + | TNFRSF19 | 407,228 | Tumor necrosis factor receptor |
| | | | | | superfamily member 19 precursor |
| | | | | | (Toxicity and JNK inducer) (TROY) |
| 23,361 | 23,369 | + | PCOTH | 186,070 | Prostate Collagen Triple Helix |
| 23,362 | 23,202 | − | MIPEP | 193,884 | Mitochondrial intermediate peptidase, |
| | | | | | mitochondrial precursor (EC 3.4.24.59) |
| | | | | | (MIP) |
| 23,426 | 23,379 | − | FLJ46358 | 129,175 | Hypothetical protein FLJ46358 |
| 23,452 | 23,795 | + | SPATA13 | 0 | Spermatogenesis associated 13 |
| 23,985 | 23,893 | − | PARP4 | 337,591 | Poly (ADP-ribose) polymerase family, |
| | | | | | member 4 |
| 24,153 | 24,184 | + | ATP12A | 597,224 | ATPase, H+/K+ transporting, |
| | | | | | nongastric, alpha |
| | | | | | polypeptide |
| 24,236 | 24,352 | + | RNF17 | 680,830 | RING finger protein 17 |
| 24,396 | 24,354 | − | CENPJ | 798,951 | Centromere protein J (Centrosomal |
| | | | | | P4.1-associated protein) (LAG-3- |
| | | | | | associated protein) (LYST- |
| | | | | | interacting protein 1) |

4.2 SACS (Distance=649,642 bp)

The SACS (or SACSIN) gene encodes 7 different transcripts through alternate splicing. The Sacsin protein is highly expressed in the central nervous system, skeletal muscle and at low levels in the pancreas. The presence of heat-shock domains suggests a function for sacsin in chaperone-mediated protein folding (Engert et al. 2000). Defects in SACS are the cause of autosomal recessive spastic ataxia of Charlevoix-Saguenay (ARSACS). ARSACS is an early onset neurodegenerative disease with high prevalence in the Charlevoix-Saguenay Lac-Saint-Jean region of Quebec. It is characterised by absent sensory-nerve conduction, reduced motor-nerve velocity and hypermyelination of retinal-nerve fibres.
Engert, J. C.; Berube, P.; Mercier, J.; Dore, C.; Lepage, P.; Ge, B.; Bouchard, J.-P.; Mathieu, J.; Melancon, S. B.; Schalling, M.; Lander, E. S.; Morgan, K.; Hudson, T. J.; Richter, A (2000) ARSACS, a spastic ataxia common in northeastern Quebec, is caused by mutations in a new gene encoding an 11.5-kb ORF. Nature Genet. 24: 120-125, 2000.

4.3 TNFRSF19 (Distance=407,228 bp)

TNFRSF19 encodes 5 distinct isoforms through alternate splicing. The protein encoded by this gene is a member of the TNF-receptor superfamily. This receptor is highly expressed during embryonic development. It has been shown to interact with TRAF family members, and to activate JNK signalling and NF-kB pathway when overexpressed in cells (Eby et al. 2000; Kojima et al. 2000). Although it lacks a death domain, this receptor is capable of programmed cell death by a caspase-independent mechanism (Wang et al 2003). TNFRSF19 also plays a role in axonal regeneration.

Myelin-associated inhibitory factors (MAIFs) are inhibitors of CNS axonal regeneration following injury. The Nogo receptor complex, composed of the Nogo-66 receptor 1 (NgR1), neurotrophin p75 receptor (p75), and LINGO-1, represses axon regeneration upon binding to these myelin components. Expression of p75 to only certain types of neurons and its temporal expression during development suggests that other receptors are involved in the NgR1 complex. TNFRSF19 is broadly expressed in postnatal and adult neurons, is able to bind to NgR1 and can replace p75 in the p75/NgR1/LINGO-1 complex to activate the GTPase RhoA, which rigidifies the actin cytoskeleton causing growth cone collapse, in the presence of myelin inhibitors (Park et al 2005; Shao et al 2005).
Eby M T, Jasmin A, Kumar A, Sharma K, Chaudhary P M. (2000) TAJ, a novel member of the tumor necrosis factor receptor family, activates the c-Jun N-terminal kinase pathway and mediates caspase-independent cell death. J Biol Chem. 2000 May 19; 275(20):15336-42.
Kojima, T., Morikawa, Y., Copeland, N. G., Gilbert, D. J., Jenkins. N. A., Senba, E. and Kitamura, T. (2000). TROY, a newly identified member of the tumor necrosis factor receptor superfamily, exhibits a homology with Edar and is expressed in embryonic skin and hair follicles. J. Biol. Chem. 275, 20742-20747
Park J B, Yiu G, Kaneko S, Wang J, Chang J, He X L, Garcia K C, He Z. (2005) A TNF receptor family member, TROY, is a coreceptor with Nogo receptor in mediating the inhibitory activity of myelin inhibitors. Neuron. 2005 Feb. 3; 45(3):345-51
Shao Z, Browning J L, Lee X, Scott M L, Shulga-Morskaya S, Allaire N, Thill G, Levesque M, Sah D, McCoy J M, Murray B, Jung V, Pepinsky R B, Mi S. (2005) TAJfTROY, an orphan TNF receptor family member, binds Nogo-66 receptor 1 and regulates axonal regeneration. Neuron. 2005 Feb. 3; 45(3):353-9.
Wang Y, Li X, Wang L, Ding P, Zhang Y, Han W, Dalong M. (2004) An alternative form of paraptosis-like cell death, triggered by TAJfTROY and enhanced by PDCD5 overexpression J Cell Sci. 2004 Mar. 15; 117(Pt 8): 1525-32.

4.4 PCOTH (Distance=186,070 bp)

PCOTH or Prostate collagen triple helix encodes 3 different transcripts and putatively 2 different protein products. PCOTH expression is limited to the testis and prostate, with significantly elevated expression in prostate cancer cells and their precursors, prostatic intraepithelial neoplasia (Ashida et al., 2004). Overexpression of PCOTH in healthy cells resulted in increased rate of cell growth/division (Anazawa et al., 2005). Conversely, PCOTH siRNA in prostatic tumour cells attenuated cell growth. PCOTH expression in prostate cancer cells was found to be associated with elevation of TAF-1 B phosphorylation. TAF-1 B (or SET) was first identified as a partner of the fusion gene in acute undifferentiated leukemia as set-can gene and was shown to be a multitasking protein such as a potent inhibitor of protein phosphatase 2A, a target of granzyme A, an inhibitor of histone acetyltransferase, and also a regulator of cell cycle transition, indicating that TAF-1β is a modulator of cell growth and proliferation (see refs 22-29 cited in Anazawa et al., 2005). PCOTH may somehow regulate the phosphorylation/activation of TAF-1β. Whereas TAF-1β is ubiquitously expressed in various tissues, PCOTH expression is exclusively observed in testis, prostate, and prostate tumors. Hence, PCOTH might serve as a prostate-, testis-, or prostate cancer-specific modulator in regulating phosphorylation of TAF-1β, with overexpression of PCOTH resulting in hyperphosphorylation of TAF-1β and thus promoting cell viability.
Anazawa Y, Nakagawa H, Furihara M, Ashida S, Tamura K, Yoshioka H, Shuin T, Fujioka T, Katagiri T, Nakamura Y. (2005) PCOTH, a novel gene overexpressed in prostate cancers, promotes prostate cancer cell growth through phosphorylation of oncoprotein TAF-Ibeta/SET. Cancer Res. June 1; 65(11):4578-86.
Ashida S, Nakagawa H, Katagiri T, et al. Molecular features of the transition from prostatic intraepithelial neoplasia (PIN) to prostate cancer: genome-wide geneexpression profiles of prostate cancers and PINs. Cancer Res 2004; 64:5963-72.

4.5 MIPEP (Distance=193,884 bp)

MIPEP or the Mitochondrial intermediate peptidase gene encodes 6 different isoforms. The protein performs the final step in the cleavage of specific classes of nuclear-encoded proteins targeted to the mitochondrial matrix or inner membrane (Isaya et al 1991). These proteins include (i) subunits of pyridine- and flavin-linked dehydrogenases; (ii) iron-sulfur cluster-containing proteins and other nucleus-encoded subunits of respiratory chain complexes; (iii) proteins required for replication and expression of mitochondrial DNA; and (iv) ferrochelatase, the enzyme that catalyzes iron attachment in the last step of heme synthesis (Branda and Isaya 1995, Chew 1997). This suggests that MIPEP is important for oxidative metabolism. MIPEP is also thought to have a role in mitochondrial iron homeostasis and perhaps a modulatory effect on clinical severity in the neurodegenerative disorder Friedrich's Ataxia (Branda 1999).
Branda S S, Isaya G. (1995) Prediction and identification of new natural substrates of the yeast mitochondrial intermediate peptidase. J. Biol. Chem. 270 27366-27373.
Branda. S S, Yang Z, Chew A, Isaya G. (1999) Mitochondrial intermediate peptidase and the yeast frataxin homolog together maintain mitochondrial iron homeostasis in Saccharomyces cerevisiae. Hum. Molec. Genet. 8: I 099-1110.
Chew A. Buck E A, Peretz S, Sirugo G. Rinaldo P, Isaya G. (I 997) Cloning, expression, and chromosomal assignment of the human mitochondrial intermediate peptidase gene (MIPEP). Genomics 40: 493-496.
Isaya G, Kalousek F, Fenton W A, Rosenberg L E. (1991) Cleavage of precursors by the mitochondrial processing peptidase requires a compatible mature protein or an intermediate octapeptide. J. Cell Biol. 113 65-76.

4.6 FLJ46358 (Distance=129,175 bp) The hypothetical protein FLJ46358 covers only 47 kb of the genome. No literature could be found for hypothetical protein FLJ46358. It was defined by 7 cDNNEST clones (from pooled pancreas and spleen, astrocytoma cell line, testis and hippocampus). It contains no protein domain or characteristic Psort motif. It is predicted to localise in the cytoplasm by Psort. Sequence homology failed to reveal any more information.

4.7 PARP4 (Distance=337,591 bp)

Poly(ADP-ribose) polymerase-4 (PARP4) catalyses the transfer of ADP-ribose moieties derived from NAO+ to various acceptor proteins including PARP4 itself. This poly(ADP-ribosyl)ation of proteins is drastically stimulated upon binding of the PARP4 DNA-binding domain (DBD) to single or double-strand breaks of DNA and is generally accepted that PARP plays an active role in the cellular recovery from DNA damage (Lindahl et al., 1995). Furthermore, it has been described that

PARP4 is quantitatively cleaved during Fas ligand- or DNA damage-induced apoptosis by caspase-3 (Tewari et al., 1995; Nicholson et al., 1995). Inhibition of PARP4 leads to genetic instability following DNA damage. Hela cells with constitutitive overexpression of dominant negative PARP4 shows reduced tumour formation in nude mice (Hans et al., 1999). This most likely follows from increased tumour cell apoptosis in vivo. PARP4 also exists in the cytoplasm and is a constituent of vaults, cytoplasmic ribonucleoprotein particles with a barrel like twisted structure and two protruding caps (Zheng et al., 2005). Vaults comprise of three proteins; Major vault protein (MVP), PARP4 and two molecules of telomerase associated protein 1 (TEP1) together with at least 6 molecules of small untranslated RNAs. The function of vaults are unknown. However, they do are associated with chemotherapy resistance in primary tumors and various tumor cell lines, being generally recognised as a negative prognostic factor for response to chemotherapy (Mossink et al., 2003). The precise role of the vaults in chemotherapy resistance is unknown, although it has been suggested that vaults may alter intracellular anticancer drug disposition, e.g., by drug sequestration in vesicles.
Lindahl T, Satoh M S, Poirier G G, Klungland A. (1995) Post-translational modification of poly(ADP-ribose) polymerase induced by DNA strand breaks. Trends Biochem Sci. 1995 October; 20(10):405-11.
Mossink M H, van Zon A, Scheper R J, Sonneveld P, Wiemer E A. (2003) Vaults: a ribonucleoprotein particle nvolved in drug resistance? Oncogene. 2003 Oct. 20; 22(47):7458-67.
Nicholson D W, Ali A, Thornberry N A, Vaillancourt J P, Ding C K, Gallant M, Gareau Y, Griffin P R, Labelle M, Lazebnik Y A, et al. (1995) Identification and inhibition of the ICE/CED-3 protease necessary for mammalian apoptosis. Nature. 1995 Jul. 6; 376(6535):37-43.
Tewari M, Quan L T, O'Rourke K, Desnoyers S, Zeng Z, Beidler D R, Poirier G G, Salvesen G S, Dixit V M. (1995) Yama/CPP32 beta, a mammalian homolog of CED-3, is a CrmA-inhibitable protease that cleaves the death substrate poly(ADP-ribose) polymerase. Cell. 1995 Jun. 2; 81(5):801-9.
Zheng C L, Sumizawa T, Che X F, Tsuyama S, Furukawa T, Haraguchi M, Gao H, Gotanda T, Jueng H C, Murata F, Akiyama S. (2005) Characterization of MVP and VPARP assembly into vault ribonucleoprotein complexes. Biochem Biophys Res Commun. 2005 Jan. 7; 326(1):100-7.
Hans M A, Muller M, Meyer-Ficca M, Burkle A, Kupper J H. (1999) Overexpression of dominant negative PARP interferes with tumor formation of HeLa cells in nude mice: evidence for increased tumor cell apoptosis in vivo. Oncogene. 1999 Nov. 25; 18(50):7010-5.

4.8 ATP12A (Distance=597,2248P)

ATP12A encodes the alpha catalytic subunit of the ouabain-sensitive H+/K+-ATPase that catalyzes the hydrolysis of ATP coupled with the exchange of H(+) and K(+) ions across the plasma membrane. It is also responsible for potassium absorption in various tissues. Tissue expression studies show significant levels in kidney and skin, moderate levels in brain and placenta, together with low levels in colon (Pestov et al., 1998).
Pestov N B, Romanova L G, Korneenko T V, Egorov M V, Kostina M B, Sverdlov V E, Askari A, Shakhparonov M I, Modyanov N N. (1998) Ouabain-sensitive H,K-ATPase: tissue-specific expression of the mammalian genes encoding the catalytic alpha subunit. FEBS Lett. 1998 Dec. 4; 440(3):320-4.

4.9 RNF17 (Distance=680,830 bp)

Ring Finger 17 is specifically expressed in testis (Wang et al 2001) and is essential in for spermiogenesis (Pan et al. 2001). It contains a Tudor domain and RING finger motif. The Tudor domain is thought to function in RNA binding or protein interactions during RNA metabolism and/or transport (Ponting, 1997). The RING finger motif is present in many ubiquitin E3 ligases (Joazeiro and Weissman, 2000; Lorick et al., 1999). RNF17 interacts with all four members of the Mad family (Mad1, Mxi1, Mad3 and Mad4) which are basic helix-loop-helix-leucine zipper transcription factors which repress Myc responsive genes through binding to Max transcription factor (Yin et al., 1999). RNF17 was shown to activate transcription of Myc responsive genes by sequestering Mad proteins (Yin et al., 2001; Yin et al., 1999). Such redistribution of Mad protein gives a ‘Mad null’ phenotype, enhancing the sensitivity of cells to several apoptotic stimuli in the same way as c-Myc overexpression.
Joazeiro, C. A. and Weissman. A. M. (2000). RI G finger proteins: mediators of ubiquitin ligase activity. Cell I 02, 549-552.
Lorick. K. L. Jensen. J. P., Fang. S., Ong. A. M., Hatakeyama. S. and Weissman. A. M. (1999). RING fingers mediate ubiquitin-conjugating enzyme (E2)-dependent ubiquitination. Proc. Natl. Acad. Sci. USA 96. 11364-11369.
Pan J, Goodheart M, Chuma S, Nakatsuji N, Page D C, Wang P J. (2005) RNF17, a component of the mammalian germ cell nuage, is essential for spermiogenesis. Development. 2005 September; 132(18):4029-39. Epub 2005 Aug. 10.
Ponting C P. (1997) Tudor domains in proteins that interact with RNA. Trends Biochem Sci. 1997 February; 22(2):51-2.
Wang, P. J., McCarrey, J. R. Yang, F. and Page. D. C. (2001). An abundance of X-linked genes expressed in spermatogonia Xat. Genet. 27, 422-426.
Yin, X. Y., Grove, L. E. and Prochownik, E. V. (2001). Mmip-2/R.nf-17 enhances c-Myc function and regulates some target genes in common with glucocorticoid hormones. Oncogene 20. 2908-2917.
Yin, X. Y. Gupta, K., Han, W. P., Levitan, E. S. and Prochownik, E. V. (1999). Mmip-2. a novel RING finger protein that interacts with mad members of the Myc oncoprotein network. Oncogene 18. 6621-6634.

4.10 CENPJ (Distance=798,951 bp)

Centromere protein J is associated with the gamma-tubulin complex and was initially identified by virtue of its interaction with the cytoskeletal protein 4.IR-135 (Hung et al., 2000). Although CPAP appears to be a component of the centrosomal complex, the majority of CPAP is found in soluble fractions, mainly in the cytoplasm and a small portion in the nucleus (Hung, 2000; Peng 2002). CPAP, which binds to STATS, translocates from the cytoplasm to the nucleus in response to prolactin-mediated activation of the JAK-STAT pathway and enhances STATS-dependent transcription (Peng 2002). CENPJ is also a coactivator of NF-kB that binds to the N-terminal region of RelA, possibly activating transcription through CBP (Koyanagi et al., 2005). NF-kB is a transcription factor important for various cellular events such as inflammation, immune response, proliferation, and apoptosis.
Hung L Y, Tang C J, Tang T K. (2000) Protein 4.1 R-135 interacts with a novel centrosomal protein (CPAP) which is associated with the gamma-tubulin complex. Mol Cell Biol. 2000 October; 20(20):7813-25
Koyanagi M, Hijikata M, Watashi K, Masui O, Shimotohno K. (2005) Centrosomal P4.1-associated protein is a new member of transcriptional coactivators for nuclear factor-kappaB. J Biol Chem. 2005 Apr. 1; 280(13):12430-7. Epub 2005 Jan. 31
Peng B, Sutherland K D, Sum E Y, Olayioye M, Wittlin S. Tang T K, Lindeman G J, Visvader J E. (2002) CPAP is a novel stat5-interacting cofactor that augments stat5-mediated transcriptional activity. Mol Endocrinol. 2002 September; 16(9):2019-33.

4.11 Conclusion

There are ten genes within 1 Mb of the insertion site, but the closest FLJ46358 is already 129 Kb distant. It would be very unlikely for the expression of genes at such a distance to be disrupted by retroviral insertion. None of the ten genes are known oncogenes, although two of them have been associated with cancer in the literature (PCOTH and PARP4). Nonetheless, these two genes are 186 and 337 kb away from the insertion respectively. It would be highly unlikely and quite unprecedented for their expression to be disrupted by a retroviral insertion at such a distance. A number of the other ten genes have been associated with disease in the literature, but there is no clear way in which they could pose a risk.

5. Discussion

Inserted proviruses can induce oncogenic activation through the process of insertional mutagenesis. The question arises of whether such activation is likely in CTX0E03 cells. The conclusion from this analysis is that it is highly unlikely.

First, it is important to note significant methodological differences between the generation of CTX0E03 cells and those scenarios where oncogenic activation has arisen (Such as the French X-SCID Gene Therapy trial). In those applications, retroviral vectors were themselves the therapeutic agents and large numbers of haematological progenitor cells were infected with retroviruses. Each of these would constitute a separate insertional event. So each patient was exposed to many potentially mutagenic events. There would then follow a selective process whereby a subset of the infected progenitors would expand within the patient. Thus, there were many opportunities for oncogenic activation of genes unidentified at the outset of the trial, and a selection pressure to increase the likelihood of tumorgenicity being revealed.

The generation of CTX0E03, by contrast, involves a single insertional event into a single identified locus, with no subsequent selection. Therefore, a priori the probability of insertional mutagenesis is both considerably reduced compared to gene therapy studies, and quantifiable in advance.

In this assessment of the likelihood of oncogenic activation, the following has been observed:
the CTX0E03 cell line has the retrovirus inserted into a gene with no known oncogenic function. The nature of the insertion is unlikely to give rise to gene disruption or activation.
The insertion does not fall in the vicinity of any endogenous retroviruses, whose activation might conceivably induce oncogenic activation.
There are no other genes other than that into which the insertion falls that are sufficiently close or of sufficient oncogenic potential to raise concerns regarding oncogenic activation.

The risk of oncogenic activation as a consequence of insertional mutagenesis can never be considered to be zero, since there must remain oncogenic genes and mechanisms yet to be discovered. Nonetheless given the state of current knowledge, it appears reasonable to conclude that the risk of oncogenic activation as a consequence of insertional mutagenesis in the CTX0E03 cell line is very low.

The use of the terms “a” and “an” and “the” and similar terms in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All citations and referenced documents are incorporated by reference in their entirety, as if each individual disclosure had been separately and expressly incorporated.

1. A mammalian cell comprising a genetic modification within the SPATA13 gene on chromosome 13q12.12, wherein:
(i) the cell is not a CTX0E03 cell; and/or
(ii) the genetic modification is not insertion of a cMyc-ER transgene.

2. A cell according to claim 1, wherein the genetic modification is an insertion.

3. A cell according to claim 1, wherein the genetic modification is an integrated transgene.

4. A cell according to claim 1, wherein the cell is a human cell or an animal cell.

5. A cell according to claim 1, wherein the cell is a stem cell, a terminally differentiated cell, an immune cell, a T cell, a B cell or a Schwann cell.

6. A cell according to claim 5, wherein the cell is a stem cell, a neural stem cell, a neural stem cell from foetal cortical tissue, from a neural stem cell line, or a mesenchymal stem cell, or an iPS cell.

7. A cell according to claim 6, wherein the genetic modification is not insertion of a cMyc-ER transgene, and wherein the cell is a CTX0E03 cell engineered to replace the cMycER-transgene with a different transgene.

8. A cell according to claim 1, wherein the cell is:
a. a Schwann cell having a cMycER transgene inserted within the SPATA13 gene on chromosome 13q12.12; or
b. a T cell having a chimeric antigen receptor inserted within the SPATA13 gene on chromosome 13q12.12.

9. A cell according to claim 1, wherein the cell is for use in a therapy.

10. A cell according to claim 1, wherein the cell is for use in a biotechnological process, optionally wherein the process is-the-comprises production of a stem cell, protein or microparticle.

11. A pharmaceutical composition comprising a cell according to claim 1.

12. A method of producing a transgenic cell comprising a stable integrated transgene, the method comprising the step of integrating the transgene at a site within the SPATA13 gene on chromosome 13q12.12.

13. A method according to claim 12, wherein the method comprises CRISPR or TALENS gene editing techniques.

14. A method according to claim 13, wherein the cell is a mammalian cell comprising a genetic modification within the SPATA13 gene on chromosome 13912.12, wherein the genetic modification is an insertion, and wherein:
(i) the cell is not a CTX0E03 cell; and/or
(ii) the genetic modification is not insertion of a cMyc-ER transgene.

15. A method according to claim 12, wherein the site is targeted using a gene therapy construct able and adapted to guide the integration of the transgene at the site following administration to a human or animal.

16. A cell obtained or obtainable from the method of claim 12.

17. A nucleic acid molecule able and adapted to guide an insertion of a transgene at a site within the SPATA13 gene on chromosome 13q12.12 following administration to a human or animal.

18. A ee nucleic acid according to claim 17, wherein the nucleic acid is for use in a therapy.

19. A nucleic acid for use according to claim 18, wherein the therapy is treatment of a disease or inherited condition.

20. A nucleic acid for use according to claim 19, wherein the therapy is treatment of cancer.

21. A cell according to claim 2, wherein the insertion is:
a. within an intron of the SPATA13 gene;
b. within the third intron of the SPATA13 gene;
c. within third intron of a cDNA clone with Genbank accession number BX648244;
d. on chromosome 13q12.12 anywhere between nucleotides 24,083,250-400 bp from the P-terminus;
e. on chromosome 13q12.12 anywhere between nucleotides 24,083,300-350 bp from the P-terminus;
f. on chromosome 13q12.12 anywhere between nucleotides 24,083,325-335 bp from the P-terminus; or
g. on chromosome 13q12.12 between nucleotides −24,083,331-24,083,332 bp from the P-terminus.
